(19) United States
(12) Patent Application Publication
(10) Pub. No.: US 2022/0285020 A1
(43) Pub. Date: Sep. 8, 2022

(54) APPLICATION CONFIGURATION BY A PATIENT CLINICIAN WITHOUT PHYSICAL ACCESS TO PATIENT DEVICE

(71) Applicant: Medtronic, Inc., Minneapolis, MN (US)

(72) Inventor: Christopher D. Koch, Minneapolis, MN (US)

(21) Appl. No.: 17/194,018

(22) Filed: Mar. 5, 2021

(51) Int. Cl. G16H 40/40; G16H 10/60; G16H 40/67; G06F 21/62; H04W 48/10; A61N 1/372

(52) U.S. Cl. CPC G16H 40/40; G16H 10/60; G16H 40/67; G06F 21/6245; H04W 48/10; A61N 1/37254; G06F 8/60

(57) ABSTRACT

An example patient device includes memory configured to store a monitoring application, communication circuitry configured to communicate with at least one of a server or another device, and processing circuitry communicatively coupled to the memory and the communication circuitry. The processing circuitry is configured to receive an advertising token from the server. The processing circuitry is configured to wirelessly advertise for a connection using the advertising token. The processing circuitry is configured to, in response to the wirelessly advertising, receive patient-specific information relating to a patient associated with the patient device from the another device and configure the monitoring application to include the patient-specific information.

### TECHNICAL FIELD

This disclosure relates generally to wirelessly configuring an application on a patient device, and more specifically, to wirelessly configuring a monitoring application on a patient device which may monitor physiological parameters sensed by an implantable medical device or otherwise allow the patient device to interact with the implantable medical device.

### BACKGROUND

Implantable medical devices (IMDs) may be surgically implanted in a patient to monitor one or more physiological parameters of the patient and/or deliver therapy to suppress one or more symptoms of the patient. For example, an IMD may comprise a cardiac monitor, be configured to deliver cardiac pacing or another stimulation therapy to the patient, and/or be configured to terminate tachyarrhythmia by delivery of high energy shocks. A clinician or patient may use an external computing device to retrieve information collected by the IMD and/or to configure or adjust one or more parameters of the therapy provided by the IMD. Typically, the external computing device connects to the IMD via a wireless connection. In some examples, a wireless connection is established between the external computing device and the IMD using a personal area networking technology, such as the Bluetooth® or Bluetooth® low energy (BLE) wireless protocol.

Implantable medical device manufacturers and/or healthcare providers may provide remote monitoring services which may communicate wirelessly with a patient or clinician external computing device through, for example, an application which may reside on the external computing device via the Internet or cellular network. The remote monitoring service may monitor the physiological parameters being sensed by the IMD and being sent to the external computing device and send alerts to the patient's external computing device and/or a clinician external computing device relating to changes in the physiological parameters, through, for example, a voice message, a short message service (SMS) text, a page, or an email.

### SUMMARY

In general, the disclosure describes techniques for a clinician to configure a monitoring application on a patient external computing device (also referred to herein as a patient device) without physically contacting the patient device. This disclosure also describes techniques for a clinician to securely establish an account with a remote monitoring service for the patient without physically contacting the patient device.

Because the IMD is implanted within the patient, a clinician or a patient uses an external computing device to configure or control the monitoring and/or therapy provided by the IMD over a wireless connection. The clinician or patient may additionally use a remote monitoring service to monitor physiological parameters being sensed by the IMD via the external computing device.

Some patients that have an IMD or that are receiving an IMD may be in relatively poor health and/or may be unfamiliar with configuring a monitoring application that may be downloaded onto a patient device. Techniques are disclosed herein for enabling a clinician to facilitate the configuration of a monitoring application on a patient device without physically contacting the patient device. For example, a clinician may use a clinician external computing device (also referred to herein as a clinician device) to wirelessly communicate with the IMD, the patient device, and/or the remote monitoring service to facilitate the configuring of the monitoring application on the patient device and/or create an account with the remote monitoring service.

By enabling a clinician to facilitate the configuration of a monitoring application on a patient device and/or the creation of an account with a remote monitoring service, the techniques of this disclosure may reduce the occurrence of errors in configuring the monitoring application and/or setting up the account and reduce the risk of legal liability that may come with a clinician physically contacting the patient device. Additionally, the techniques of this disclosure may enhance the security of the configuration and the setting up of the account, thereby protecting sensitive information, such as personal healthcare records and personally identifiable information of the patient.

In one example, this disclosure describes a method including receiving, by a patient device, an advertising token from a server; wirelessly advertising for a connection, by the patient device, using the advertising token; in response to the wirelessly advertising, receiving, by the patient device and from another device, patient-specific information relating to a patient associated with the patient device; and configuring, by the patient device, a monitoring application to include the patient-specific information.

In another example, this disclosure describes a patient device including memory configured to store a monitoring application, communication circuitry configured to communicate with at least one of a server or another device, and processing circuitry communicatively coupled to the memory and the communication circuitry, the processing circuitry being configured to: receive an advertising token from the server via the communication circuitry; wirelessly advertise for a connection using the advertising token via the communication circuitry; in response to the wirelessly advertising, receive patient-specific information relating to a patient associated with the patient device from the another device via the communication circuitry; and configure the monitoring application to include the patient-specific information.

In another example, this disclosure describes a method including receiving, by a clinician device and from a patient device, a first advertising token; receiving, by the clinician device and from an implantable medical device, a second advertising token; transmitting, by the clinician device to a server, a first identifier associated with the patient device and a second identifier associated with the implantable medical device; receiving, by the clinician device from the server, authorization to input patient-specific information associated with a patient associated with the patient device and the implantable medical device; receiving, by the clinician device from a clinician, the patient-specific information; and transmitting, by the clinician device to another device, the patient-specific information.

In another example, this disclosure describes a clinician device including memory configured to store a monitoring application, communication circuitry configured to communicate with at least one of a server or another device, a user interface configured to accept input from a user, and processing circuitry communicatively coupled to the memory, the communication circuitry and the user interface, the processing circuitry being configured to: receive a first advertising token from a patient device via the communication circuitry; receive a second advertising token from an implantable medical device via the communication circuitry; transmit a first identifier associated with the patient device and a second identifier associated with the implantable medical device to a server via the communication circuitry; receive authorization to input patient-specific information associated with a patient associated with the patient device and the implantable medical device from the server via the communication circuitry; receive, from a clinician, the patient-specific information, via the user interface; and transmit, to the another device, the patient-specific information via the communication circuitry.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a conceptual diagram illustrating a system which may be used to monitor physiological parameters of a patient in accordance with the techniques of the disclosure.

FIG. 2 is a conceptual diagram illustrating the IMD and leads of the system of FIG. 1 in greater detail.

FIG. 3 is a block diagram illustrating an example external computing device in accordance with the techniques of the disclosure.

FIG. 4 is a block diagram illustrating an example system in accordance with one or more techniques of this disclosure.

FIG. 5 is a conceptual diagram illustrating flows within the system of FIG. 4 in accordance with one or more techniques described herein.

FIG. 6 is a flowchart illustrating an example of configuration techniques in accordance with this disclosure.

FIG. 7 is a flowchart illustrating another example of configuration techniques in accordance with this disclosure.

Like reference characters refer to like elements throughout the figures and description.

### DETAILED DESCRIPTION

Various medical devices, including implantable medical devices (IMDs) such as pacemakers, may include sensors which may sense vital physiological parameters of a patient. Such medical devices may be configured to communicate with external computing devices through secure wireless communications technologies, such as personal area networking technologies like Bluetooth® or Bluetooth® low energy (BLE) wireless protocol. For example, a patient having such a medical device may be able to send and/or receive information relating to the operation of the IMD or to physiological parameters sensed by the IMD via such secure wireless communications technologies through the use of a wireless communications device, such as a cellular phone (e.g., a smartphone), a tablet, a laptop, or the like. The wireless communications device of the patient (which also may be referred to as a patient device) may not be configured “out of the box” to send or receive information to or from the IMD. As such, the patient device may require an application which may be specifically configured to communicate with the particular type of IMD implanted in the patient.

In some examples, the monitoring application may need to be configured to interact with both an internet-based service infrastructure (such as Carelink by Medtronic of Dublin, Ireland) and the IMD. This monitoring application configuration may involve several inputs of information by the patient for proper operation of the system as a whole. However, the patient may not be able to directly input information for configuration into the patient device in a timeframe that is suitable for a business process. Further, manual entry of information into a configuration process by a person that performs this process rarely, perhaps only once or twice in a lifetime, is prone to input errors. This input process may be further complicated if the patient is in poor health or unfamiliar with configuring applications downloaded from a server. Thus, it may be desirable for someone more familiar with configuring the monitoring application to configure the monitoring application on behalf of the patient.

However, the patient device may be owned by the patient and any physical access to the patient device by another person may create legal liability issues for the person who enters in the information. For example, the person entering the information may damage the patient device or may come into possession of personal information of the patient while handling the patient device. Therefore, techniques that may be used to configure an application on a patient device, but which do not require another person to physically access the patient device, may be desirable as such techniques may be used to configure the patient device without creating such liability.

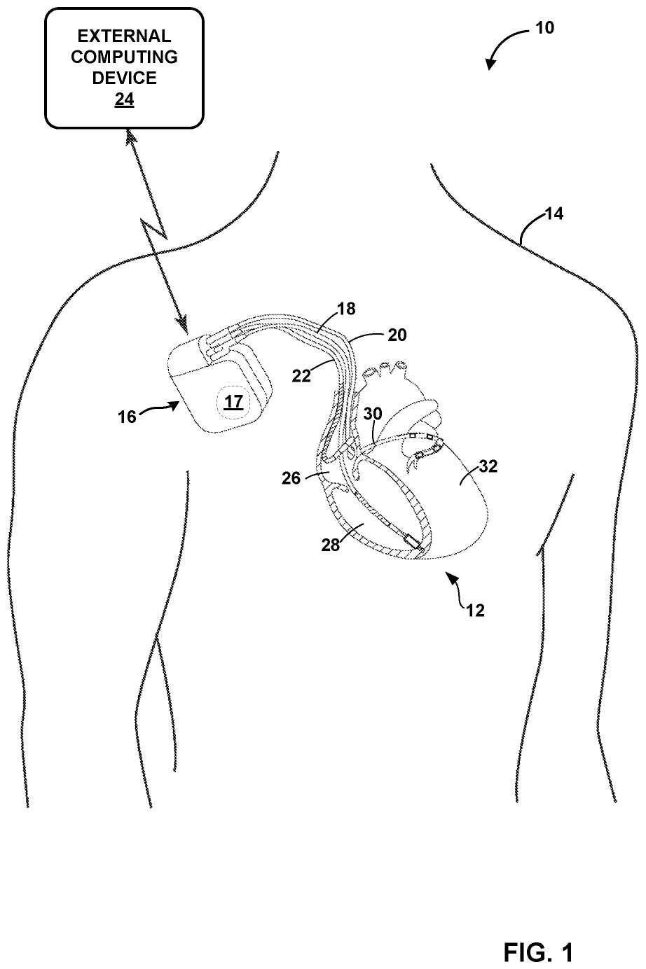

FIG. 1 is a block diagram illustrating system 10 which may be used to monitor physiological parameters of a patient in accordance with the techniques of the disclosure. As illustrated by example system 10 in FIG. 1, IMD 16 may, in some examples, be an implantable cardiac pacemaker, implantable cardioverter/defibrillator (ICD), or pacemaker/cardioverter/defibrillator, for example. While primarily described herein with respect to an implantable cardiac pacemaker, the techniques of this disclosure may be used with any implantable medical device configured to communicate wirelessly with an external computing device and configured to sense physiological parameters of a patient.

IMD 16 is connected to leads 18, 20 and 22 and is communicatively coupled to external computing device 24. IMD 16 senses electrical signals attendant to the depolarization and repolarization of heart 12, e.g., a cardiac electrogram (EGM), via electrodes on one or more leads 18, 20 and 22 or the housing of IMD 16. IMD 16 may also deliver therapy in the form of electrical signals to heart 12 via electrodes located on one or more leads 18, 20 and 22 or a housing of IMD 16. The therapy may be pacing, cardioversion and/or defibrillation pulses. IMD 16 may monitor EGM signals collected by electrodes on leads 18, 20 or 22, and based on the EGM signal, diagnose, and treat cardiac episodes.

In some examples, IMD 16 includes communication circuitry 17 including any suitable circuitry, firmware, software, or any combination thereof for communicating with another device, such as external computing device 24 of FIG. 1. For example, communication circuitry 17 may include one or more processors, memory, wireless radios, antennae, transmitters, receivers, modulation and demodulation circuitry, filters, amplifiers, or the like for radio frequency communication with other devices, such as external computing device 24. IMD 16 may use such communication circuitry to, for example, transmit one or more advertisements indicating the availability of the IMD for a wireless connection during a period of time wherein IMD 16 is discoverable. Upon establishing a wireless connection to external computing device 24, IMD 16 may use communication circuitry 17 to receive data from external computing device 24 to control one or more operations of IMD 16 and/or send uplinked data, such as sensed physiological parameters, to external computing device 24.

Leads 18, 20, 22 extend into the heart 12 of patient 14 to sense electrical activity of heart 12 and/or deliver electrical stimulation to heart 12. In the example shown in FIG. 1, right ventricular (RV) lead 18 extends through one or more veins (not shown), the superior vena cava (not shown), and right atrium 26, and into right ventricle 28. Left ventricular (LV) lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

In some examples, external computing device 24 takes the form of a handheld computing device, computer workstation, networked computing device, smartphone, tablet, laptop computer or external programmer that includes a user interface for presenting information to and receiving input from a user. A user, such as a physician, technician, surgeon, electro-physiologist, other clinician, clinician, or patient, may interact with external computing device 24 to retrieve physiological or diagnostic information from IMD 16. A user may also interact with external computing device 24 to program IMD 16, e.g., select values for operational parameters of the IMD. External computing device 24 may include a processor configured to evaluate EGM signals or other physiological parameters transmitted from IMD 16 to external computing device 24.

IMD 16 and external computing device 24 may communicate via wireless communication using any techniques known in the art. Examples of communication techniques may include, for example, communication according to a personal area network technology, such as the Bluetooth® or BLE protocols. Other communication techniques are also contemplated. External computing device 24 may also communicate with one or more other external computing devices using a number of known communication techniques, both wired and wireless.

In accordance with the techniques of the disclosure, devices, systems, and techniques for facilitating a clinician to configure a monitoring application on a patient device, such as external computing device 24, and/or set up an account with a remote monitoring service without physically contacting external computing device 24 are described. These techniques may include using the personal area network technology to interact with the IMD, the patient device, and in some examples, a server which may be configured to host a remote monitoring service.

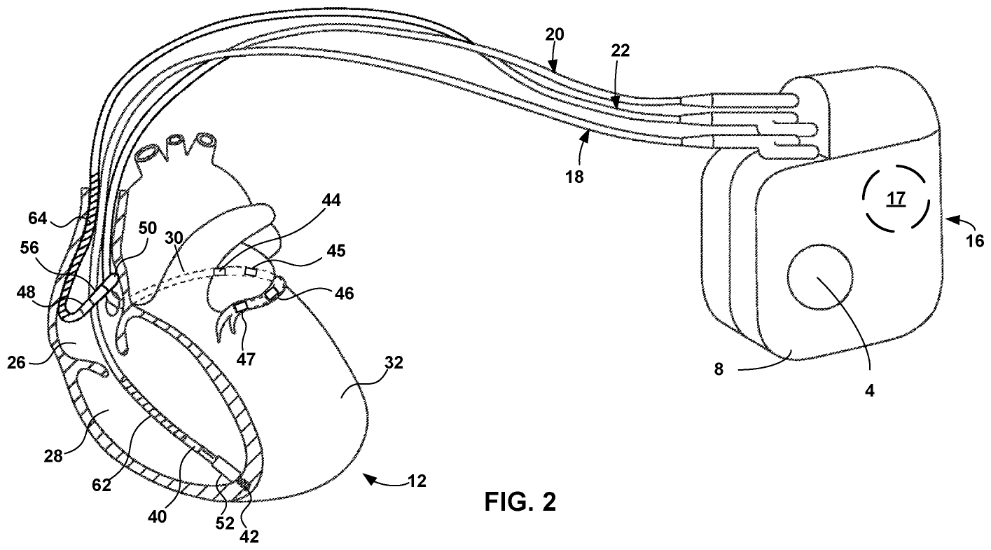

FIG. 2 is a conceptual diagram illustrating IMD 16 and leads 18, 20 and 22 of system 10 in greater detail. In the illustrated example, bipolar electrodes 40 and 42 are located adjacent to a distal end of lead 18, and bipolar electrodes 48 and 50 are located adjacent to a distal end of lead 22. In addition, four electrodes 44, 45, 46 and 47 are located adjacent to a distal end of lead 20. Lead 20 may be referred to as a quadrapolar LV lead. In other examples, lead 20 may include more or fewer electrodes. In some examples, LV lead 20 comprises segmented electrodes, e.g., in which each of a plurality of longitudinal electrode positions of the lead, such as the positions of electrodes 44, 45, 46 and 47, includes a plurality of discrete electrodes arranged at respective circumferential positions around the circumference of lead.

In the illustrated example, electrodes 40 and 44-48 take the form of ring electrodes, and electrodes 42 and 50 may take the form of extendable helix tip electrodes mounted retractably within insulative electrode heads 52 and 56, respectively. Leads 18 and 22 also include elongated electrodes 62 and 64, respectively, which may take the form of a coil. In some examples, each of electrodes 40, 42, 44-48, 50, 62, and 64 is electrically coupled to a respective conductor within the lead body of its associated lead 18, 20, 22 and thereby coupled to circuitry within IMD 16.

In some examples, IMD 16 includes one or more housing electrodes, such as housing electrode 4 illustrated in FIG. 2, which may be formed integrally with an outer surface of hermetically-sealed housing 8 of IMD 16 or otherwise coupled to housing 8. In some examples, housing electrode 4 is defined by an uninsulated portion of an outward facing portion of housing 8 of IMD 16. Other divisions between insulated and uninsulated portions of housing 8 may be employed to define two or more housing electrodes. In some examples, a housing electrode comprises substantially all of housing 8.

Housing 8 encloses a signal generator that generates therapeutic stimulation, such as cardiac pacing, cardioversion, and defibrillation pulses, as well as a sensing module for sensing electrical signals attendant to the depolarization and repolarization of heart 12. Housing 8 may also enclose a memory for storing the sensed electrical signals. Housing 8 may also enclose communication circuitry 17 for communication between IMD 16 and external computing device 24. For example, communication circuitry 17 may be configured to communicate with external computing device 24 via a personal area networking technology, such as Bluetooth® or Bluetooth® low energy (BLE) wireless protocol. In addition, or alternatively, communication circuitry 17 may be configured to communicate with external computing device 24 via another wireless technology, such as a cellular or local area network wireless technology. IMD 16 may use communication circuitry 17 for communicating with a patient device and/or a clinician device, as will be described in more detail later in this disclosure.

IMD 16 senses electrical signals attendant to the depolarization and repolarization of heart 12 via electrodes 4, 40, 42, 44-48, 50, 62, and 64. IMD 16 may sense such electrical signals via any bipolar combination of electrodes 40, 42, 44-48, 50, 62, and 64. Furthermore, any of the electrodes 40, 42, 44-48, 50, 62, and 64 may be used for unipolar sensing in combination with housing electrode 4.

The illustrated numbers and configurations of leads 18, 20 and 22 and electrodes are merely examples. Other configurations, i.e., number and position of leads and electrodes, are possible. In some examples, system 10 may include an additional lead or lead segment having one or more electrodes positioned at different locations in the cardiovascular system for sensing and/or delivering therapy to patient 14. For example, instead of or in addition to intercardiac leads 18, 20 and 22, system 10 may include one or more epicardial or extravascular (e.g., subcutaneous or substernal) leads not positioned within heart 12.

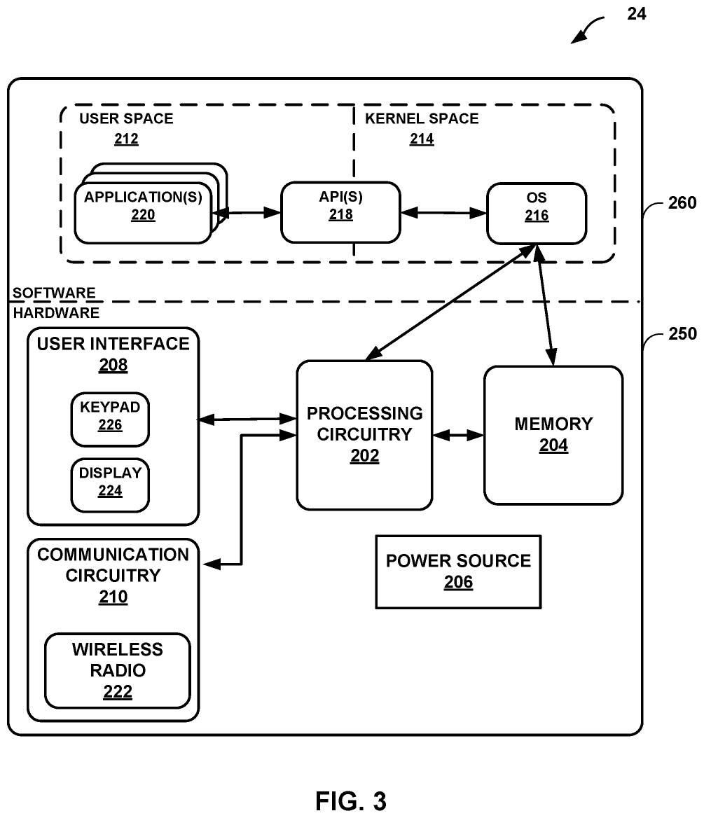

FIG. 3 is a block diagram illustrating an example external computing device 24 in accordance with the techniques of the disclosure. In some examples, external computing device 24 is an off the shelf computing device, such as a smartphone, a tablet, a laptop, or the like.

In the example of FIG. 3, external computing device 24 is described as including a hardware environment 250 and a software environment 260. However, in some examples, all or a portion of hardware environment 250 may be implemented as software. Further, in some examples, all or a portion of software environment 260 may be implemented as hardware. External computing device 24 may represent a patient device and/or a clinician device, according to the techniques of this disclosure.

Hardware environment 250 includes processing circuitry 202, memory 204, user interface 208, communication circuitry 210, and power source 206. A clinician or patient may interact with processing circuitry 202 via a user interface 208 to, for example, program therapy for patient 14. In other examples, a clinician or patient may interact with processing circuitry 202 to retrieve information from IMD 16, program a monitoring function of processing circuitry 202 or IMD 16, or for other reasons not expressly describe herein. In some examples, a clinician may interact with processing circuitry 202 of a clinician device to facilitate the configuration of a monitoring application on a patient device and/or set up an account with a remote monitoring service, according to the techniques of this disclosure.

Further, processing circuitry 202 may establish a wireless connection with IMD 16 via wireless radio 222 of communication circuitry 210. In some examples, wireless radio 222 of communication circuitry 210 may be configured to use a personal area networking technology, such as Bluetooth® or Bluetooth® low energy (BLE) wireless protocol. Additionally, or alternatively, wireless radio 222 of communication circuitry 210 may be configured to use another wireless technology, such as a cellular or local area network wireless technology. In some examples, wireless radio 222 of communication circuitry 210 may be configured to establish a wireless connection with another wireless radio 222 of another external computing device 24, such as a clinician device establishing a wireless connection with a patient device or vice versa. In some examples, wireless radio 222 of communication circuitry 210 may be configured to establish a wireless connection with a server, such as a server being a central services point for a patient monitoring service.

Processing circuitry 202 may generate information regarding the wireless connection or other information for presentation to the patient or clinician via user interface 208. User interface 208 may include display 224 and keypad 226. In some examples, keypad 226 may be a virtual keypad displayed on a touch screen. In some examples, user interface 208 may include peripheral pointing devices (not shown) for receiving input from a user. Processing circuitry 202 may include one or more microprocessors, digital signal processors (DSPs), application specific integrated circuits (ASICs), field programmable gate arrays (FPGAs), or any other equivalent integrated or discrete logic circuitry, as well as any combinations of such components. In some examples, processing circuitry 202 may be configured to perform one or more techniques of this disclosure.

External computing device 24 also includes a memory 204. Memory 204 may include program instructions that, when executed by processing circuitry 202, cause external computing device 24 to perform the functions ascribed to external computing device 24 herein. In some examples, memory 204 is random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Further, memory 204 may be implanted entirely in hardware, software, or a combination thereof.

External computing device 24 includes power source 206. Power source 206 is configured to deliver operating power to the components of external computing device 24. Power source 206 may include a battery and a power generation circuit to produce the operating power. In some examples, the battery is rechargeable to allow extended operation. Recharging may be accomplished by electrically coupling power source 206 to a cradle or plug that is connected to an alternating current (AC) outlet. In addition, recharging may be accomplished through proximal inductive interaction between an external charger and an inductive charging coil within external computing device 24. In other examples, traditional batteries (e.g., nickel cadmium or lithium ion batteries) may be used. In addition, external computing device 24 may be directly coupled to an alternating current outlet to operate.

Processing circuitry 202 and memory 204 provide an operating environment for a software stack (e.g., software environment 260) that executes one or more application(s) 220 for establishing a wireless connection to IMD 16 such that applications 220 may interact with IMD 16 to, for example, configure one or more operations, functions or applications of IMD 16, upload data from or download data to IMD 16, control therapy of IMD 16, etc. For example, the one or more applications 220 may include a monitoring application that may be used to monitor physiological parameters sensed by IMD 16 and/or to communicate the physiological parameters to a remote monitoring service. External computing device 24 partitions the virtual and/or physical address space provided by memory 204 into user space 212, allocated for running user processes, and kernel space 214, which is protected and generally inaccessible by user processes. Operating system kernel 216 executes in kernel space 214 and may include, for example, an Android™ operating system kernel available from Google, Inc., an iOS™ operating system kernel available from Apple, Inc., a Windows-based operating system kernel, available from Microsoft Corp., or a Linux- or other Unix-variant-based kernel. In other examples, operating system kernel 216 may include another type of operating system kernel not expressly described herein.

Communication circuitry 210 includes any suitable circuitry, firmware, software, or any combination thereof for communicating with another device, such as IMD 16 of FIG. 1. For example, communication circuitry 210 may include wireless radio 222, one or more antennae, transmitters, receivers, modulation and demodulation circuitry, filters, amplifiers, or the like for radio frequency communication with other devices, such as another external computing device 24, IMD 16, an access point, or a server. Under the control of processing circuitry 202, communication circuitry 210 may receive downlinked data from and send uplinked data to IMD 16 with the aid of wireless radio 222, which may include at least one antenna, which may be internal and/or external. Processing circuitry 202 may provide the data to be uplinked to IMD 16 and the control signals for wireless radio 222 within communication circuitry 210, e.g., via an address/data bus. In some examples, communication circuitry 210 may provide received data to processing circuitry 202 for processing or to memory 204 for storage.

In one example, applications 220 execute in user space 212 to communicate with IMD 16. Applications 220 may invoke one or more Application Programming Interfaces (APIs) 218 to communicate with operating system 216. APIs 218 may execute in user space 212, kernel space 214, or a combination of kernel space 214 and user space 212. For example, applications 220 may invoke a wireless communication API of APIs 218 to establish a wireless communication session with IMD 16, with another external computing device, an access point, or a server. In some examples, the wireless communication API of APIs 218 is a personal area network API, such as a Bluetooth® or BLE API. The wireless communication API of APIs 218 may provide a library of functions that, when used, cause operating system 216 to control communication circuitry 210 and wireless radio 222 of hardware environment 250 to establish the wireless communication session with IMD 16 or with another external computing device.

In some examples, operating system 216 is a version of Android™ OS and API 218 is a Bluetooth® or BLE API. Further description regarding the implementation of the Bluetooth® protocol in Android™ OS is provided in Bluetooth, Android™ Developers, available at https://developer.android.com/guide/topics/connectivity/bluetooth.html. Further description regarding the implementation of the BLE protocol in Android™ OS is provided in Bluetooth Low Energy, Android™ Developers, available at https://developer.android.com/guide/topics/connectivity/bluetooth-le.html.

In some examples, OS 216 is a version of Apple iOS™ and API 218 is a Bluetooth® or BLE API. Further description regarding the implementation of the Bluetooth protocol in Apple iOS™ is provided in About Core Bluetooth, Apple Inc., available at https://developer.apple.com/library/content/documentation/NetworkingInternetWeb/Conceptual/CoreBluetooth_concepts/AboutCoreBluetooth/Introduction.html. Further description regarding the implementation of the BLE protocol in Apple iOS™ is provided in Core Bluetooth, Apple Developer Documentation, Apple Inc., available at https://developer.apple.com/documentation/corebluetooth. In other examples, OS 216 is a Windows-based operating system kernel, available from Microsoft Corp., a Linux- or other Unix-variant-based kernel, or another type of operating system kernel not expressly described herein.

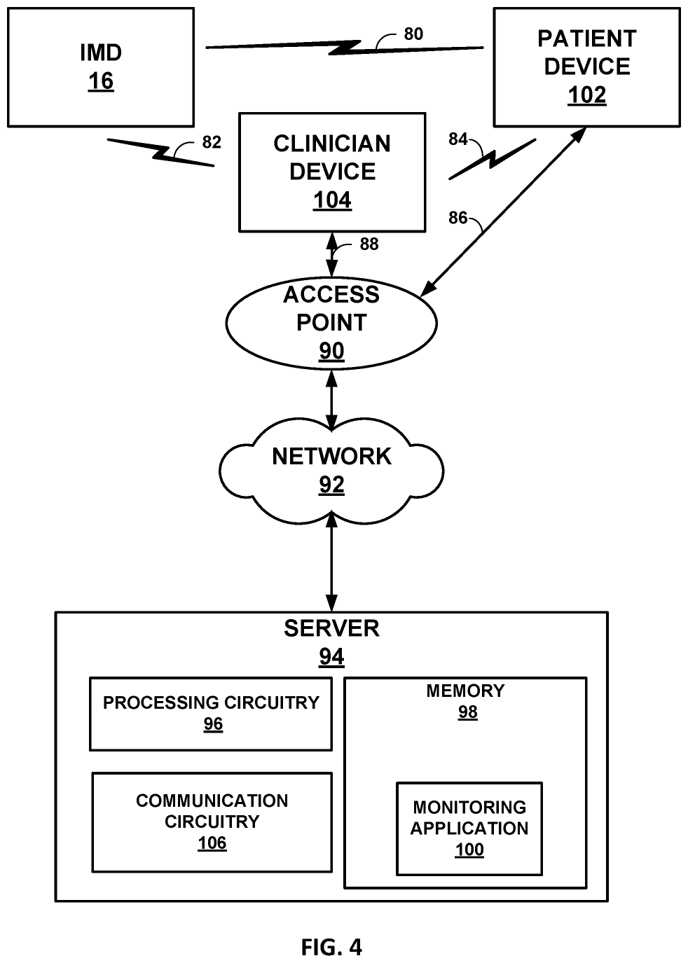

FIG. 4 is a block diagram illustrating an example system according to the techniques of this disclosure. In this example, IMD 16 may use communication circuitry 17 (of FIG. 2) to communicate with patient device 102 and with clinician device 104 via wireless connection 80 and wireless connection 82, respectively. Patient device 102 may be an example of external computing device 24. Clinician device 104 may also be an example of external computing device 24. In some examples, wireless connection 80 and wireless connection 82 may be Bluetooth® or BLE connections. In some examples, patient device 102 and clinician device 104 may also communicate via wireless connection 84. In some examples, wireless connection 84 may be a Bluetooth® or BLE connection. Patient device 102 and/or clinician device 104 may also communicate with server 94, via access point 90 and network 92 via wireless connection 86 and/or wireless connection 88, respectively. In some examples, wireless connection 86 and/or wireless connection 88 may be wireless local area network connections. In such examples, access point 90 may be a wireless local area network access point. In other examples, wireless connection 86 and/or wireless connection 88 may be cellular or wide area network connections. In the case where wireless connection 86 or wireless connection 88 is a cellular or wide area network connection, access point 90 may represent a cellular or wide area network access point such as a base station, femto cell, pico cell, or other device configured to provide access to a cellular or wide area network.

As discussed above, IMD 16 may be configured to transmit sensed physiological parameters, such as an EGM signal, or other sensed physiological parameters, to a monitoring application on patient device 102. For example, the monitoring application may, at initial startup on patient device 102, register via a web services interface to server 94, which may be a central service point of a patient monitoring service. The monitoring application may receive a BLE peripheral token from server 94 to advertise over BLE. A clinician application on clinician device 104 may be in BLE range of patient device 102 and IMD 16 which may be implanted in patient 14. IMD 16 may also be advertising via BLE. As the clinician should know the data source name (DSN) of IMD 16, the clinician may enter the DSN into the clinician application on clinician device 104.

The clinician application may send the DSN and the universally unique identifiers (UUIDs) from patient device 102 and IMD 16 that clinician device 104 has seen advertised, and the patient application UUID to server 94. Server 94 may determine if the DSN and device UUIDs are paired, if the token advertisement is one that the central service point authored, and if the token advertisement is valid. For example, it may be desirable to correlate the UUID and the DSN to prevent or reduce the risk of unauthorized users creating or accessing accounts with the central service point, e.g., sever 94. Based on the determination that the DSN and device UUIDs are paired and the advertisement is valid and was authored by the central service point, server 94 may permit the clinician to input patient specific information in the clinician application on clinician device 104 and send the patient specific information to server 94. For example, the clinician may enter a patient's name, date of birth, email, phone number, or other information relating to the patient and set up an account on server 94 for the patient. Server 94 may then push the patient specific information to the patient device application on patient device 102 via wireless connection 86, thereby configuring the patient device application to communicate with both IMD 16 and server 94.

These techniques may have the advantage of not requiring patient interaction for paring to the clinician application. By not requiring patient interaction for pairing, the patient may download the application several days before a procedure, office visit, or other in person meeting with the clinician and allow the clinician to configure the patient application when the clinician and patient are in relatively close proximity to one another with minimal involvement of the patient.

In another example, the patient application may use a predefined peripheral BLE advertisement token. Patient device 102 may advertise the predefined token and clinician device 104 and clinician application may detect this predefined token. In this example, patient 14 may interact with a patient device application user interface when in close proximity of the clinician application, for example, within BLE communication range. In this example, the patient interaction with the user interface may be reflected in realtime on the clinician application, thereby allowing the clinician application to create a pair with the patient application instance. This would then allow the patient information to be entered into the patient application via the clinician application, but the clinician application and the patient application would use a personal area network technology, such as BLE, for the communications therebetween. As the clinician application may be an authorized component of the system, the clinician application may enter information directly into patient device 102, and then permit the patient to start the association with server 94. For security purposes, the predefined peripheral BLE advertisement token may be a rolling key generated value to prevent or reduce the risk of replay attacks and spoofing.

In some examples, the techniques of this disclosure do involve patient 14, but only initially, for example, to download the monitoring application and/or to assist in pairing patient device 102 and clinician device 104, by for example, turning on BLE on patient device 102. These techniques, however, may not require the use of server 94 for coordination between the patient application on patient device 102 and the clinician application on clinician device 104.

In some examples, rather than server 94 sending a link to the account to patient device 102, clinician device 104 may send the link including the configuration information to patient device 102 via a personal area network technology, such as BLE, a wireless local area network, cellular network, or a wired connection between clinician device 104 and patient device 102. In some examples, the link may be included in an email, a short message service (SMS) text, the monitoring application, or the like.

In some examples, server 94 may be configured to provide a remote patient monitoring service. For example, server 94 may be configured to monitor physiological parameters sensed by IMD 16. These parameters may be sent by IMD 6 to the monitoring application on patient device 102. The monitoring application may then provide these parameters to server 94 via a wireless connection such as those described herein. Server 94 may monitor such parameters to determine if the parameters indicate a need for further care, clinician intervention, and the like. Server 94 may send an alert to patient device 102 and/or clinician device 104 when such further care, clinician intervention, or the like is needed. This alert may be sent via a website, a voice message, a page, an SMS message, an email message, an alert in the monitoring application, or the like.

Server 94 may also be configured to transmit monitoring application 100 to patient device 102 based on a request from patient device 102 and/or clinician device 104. In some examples, server 94 may be configured as a secure storage site for data that has been collected from IMD 16, and/or patient device 102, such as the sensed physiological parameters. In some examples, server 94 may assemble data in web pages or other documents for viewing by trained professionals, such as clinicians, via clinician device 104. One or more aspects of the illustrated system of FIG. 4 may be implemented with general network technology and functionality, which may be similar to that provided by the Medtronic CareLink® Network developed by Medtronic plc, of Dublin, Ireland.

Server 94 may include processing circuitry 96. Processing circuitry 96 may include fixed function circuitry and/or programmable processing circuitry. Processing circuitry 96 may include any one or more of a microprocessor, a controller, a DSP, an ASIC, an FPGA, or equivalent discrete or analog logic circuitry. In some examples, processing circuitry 96 may include multiple components, such as any combination of one or more microprocessors, one or more controllers, one or more DSPs, one or more ASICs, or one or more FPGAs, as well as other discrete or integrated logic circuitry. The functions attributed to processing circuitry 96 herein may be embodied as software, firmware, hardware or any combination thereof. In some examples, processing circuitry 96 may perform one or more techniques described herein including those techniques ascribed to server 94.

Server 94 may include communication circuitry 106. Communication circuitry 106 may function similarly to communication circuitry 210 of external computing device 24 of FIG. 3. For example, communication circuitry 106 may be configured to use a wireless technology, such as a cellular, a local area network wireless technology, or a personal area network wireless technology. In some examples, communication circuitry 210 may be configured to establish a wireless connection with wireless radio 222 of external computing device 24.

Server 94 may include memory 98. Memory 98 includes computer-readable instructions that, when executed by processing circuitry 96, may cause processing circuitry 96 to perform various functions attributed processing circuitry 96 herein. Memory 98 may include any volatile, non-volatile, magnetic, optical, or electrical media, such as RAM, ROM, NVRAM, EEPROM, flash memory, or any other digital media. In some examples, memory 98 may also store monitoring application 100, which may be an example of a monitoring application of one or more applications 220 of FIG. 3. Monitoring application 100 may be available for download, for example, to patient device 102 through network 92, access point 90 and wireless connection 86.

In some examples, clinician device 104 or another device located with a clinician, may program, receive alerts from, and/or interrogate IMD 16. For example, the clinician may access physiological parameters sensed by IMD 16 through clinician device 104, even when patient 14 is in between clinician visits, to check on a status of a medical condition, such as heart failure, from server 94, for example, when server 94 is operating a remote monitoring service. In some examples, the clinician may enter instructions for a medical intervention for patient 14 into an app in clinician device 104, such as based on a status of a patient condition determined by IMD 16, patient device 102, server 94, or any combination thereof, or based on other patient data known to the clinician. Clinician device 104 then may transmit the instructions for medical intervention to server 94 or patient device 102. For example, such instructions for medical intervention may include an instruction to change a drug dosage, timing, or selection, to schedule a visit with the clinician, or to seek medical attention. In further examples, clinician device 104 may generate an alert to patient 14 based on a status of a medical condition of patient 14 determined by IMD 16, which may enable patient 14 proactively to seek medical attention prior to receiving instructions for a medical intervention. In this manner, patient 14 may be empowered to take action, as needed, to address their medical status, which may help improve clinical outcomes for patient 14.

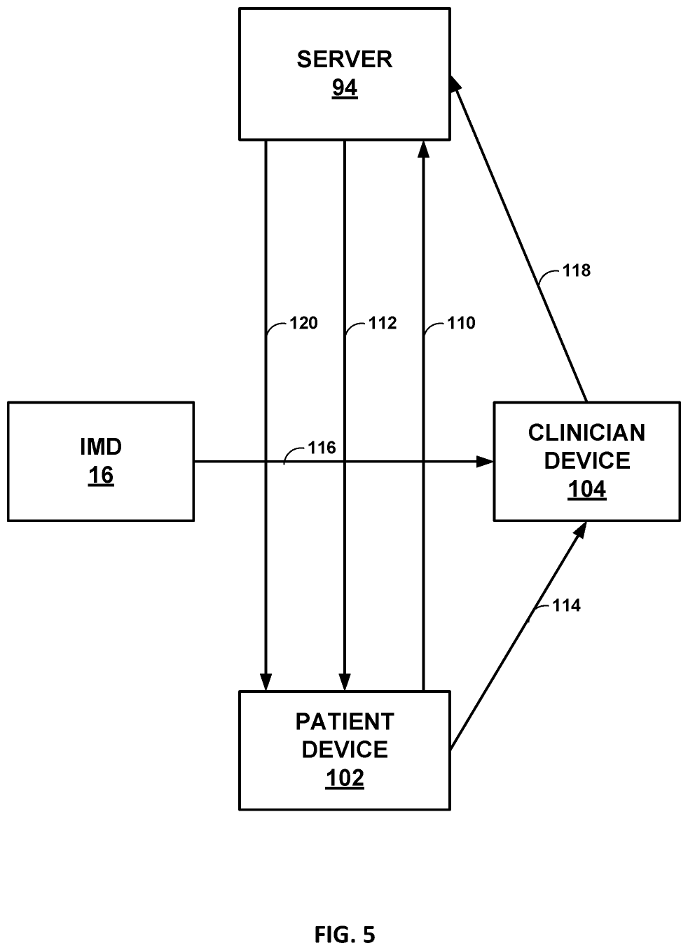

FIG. 5 is a conceptual diagram illustrating communications between devices according to the techniques of this disclosure. For example, monitoring application on patient device 102 may register with server 94 as requiring a UUID and may provide server 94 with a monitoring application instance (110). Server 94 may provide to the monitoring application instance on patient device 102 a UUID to broadcast as a peripheral (112). The monitoring application instance on patient device 102 may advertise the received UUID which may be received by clinician device 104 (114). IMD 16 may also advertise a UUID which may also be received by clinician device 104 (116). Clinician device 104 may send both the application instance's UUID and IMD 16's UUID to server 94. Server 94 may associate the application instance's UUID with IMD 16's UUID (118). Server 94 may send an invitation, e.g., via email, SMS (e.g., text) message, through the monitoring application, or the like, to patient device 102 with credentials for patient 14 to use to log into an account on server 94 (120).

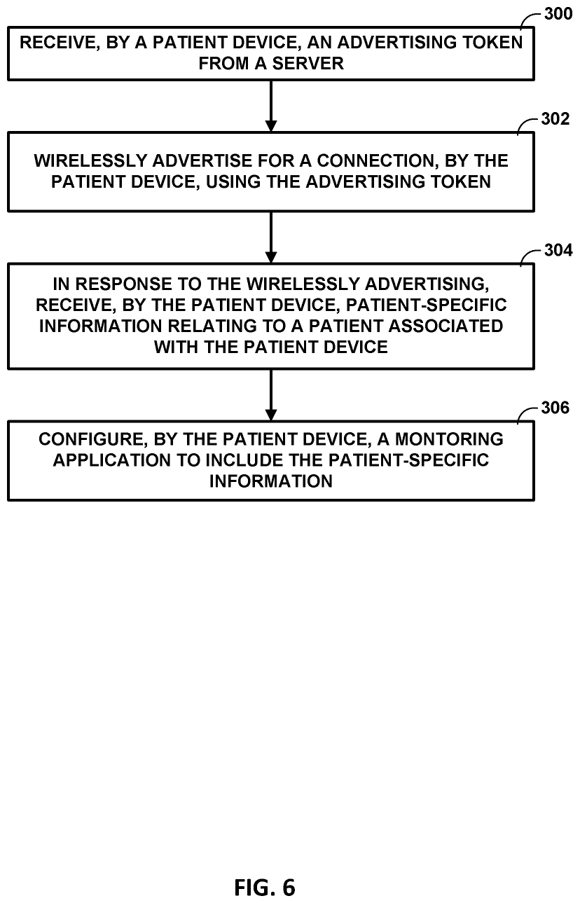

FIG. 6 is a flowchart illustrating an example of configuration techniques in accordance with this disclosure. Patient device 102 may receive an advertising token from server 94 (300). For example, patient device 102 may register with server 94 via communication circuitry 106 and communication circuitry 210 and request an advertising token, such as a BLE peripheral token or a UUID, from server 94. Server 94 may transmit the advertising token to patient device 102 via communication circuitry 106 and communication circuitry 210. Patient device 102 may register with server 94 prior to or while patient device is in close proximity (e.g., within the range of a personal area network) to clinician device 104. Patient device 102 may receive the advertising token via communication circuitry 210.

Patient device 102 may wirelessly advertise for a connection using the advertising token (302). For example, communication circuitry 210 may advertise for a BLE connection using the advertising token.

In response to the wirelessly advertising, patient device 102 may receive patient-specific information relating to a patient associated with patient device 102 from another device (304). In some examples, the another device is server 94. In other examples, the another device is clinician device 104.

For example, clinician device 104 may receive the wireless advertisement from patient device 102 and a wireless advertisement from IMD 16. Clinician device 104 may send a DSN of IMD 16 and/or a UUID associated with IMD 16 and a UUID associated with patient device 102 to server 94. In some examples, server 94 may verify that the UUID from patient device 102 is authorized and associate IMD 16 with patient device 102. Server 94 may send an authorization to clinician device 104 to enable clinician device 104 to enter patient-specific information in an instance of the monitoring application running on clinician device 104. In some examples, clinician device 104 may transmit the patient-specific information to server 94 and server 94 may push the patient-specific information to patient device 102 via communication circuitry 106. In other examples, clinician device 104 may connect with patient device 102 and push the patient-specific information to patient device 102 directly via communication circuitry 210. For example, clinician device 104 may connect with patient device 102 via a personal area networking technology.

Patient device 102 may configure the monitoring application to include the patient-specific information (306). For example, patient device 102 may populate the monitoring application with the patient-specific information. In some examples, patient device 102 may download the monitoring application from server 94 via communication circuitry 210. In some examples, the advertising token is a BLE peripheral token.

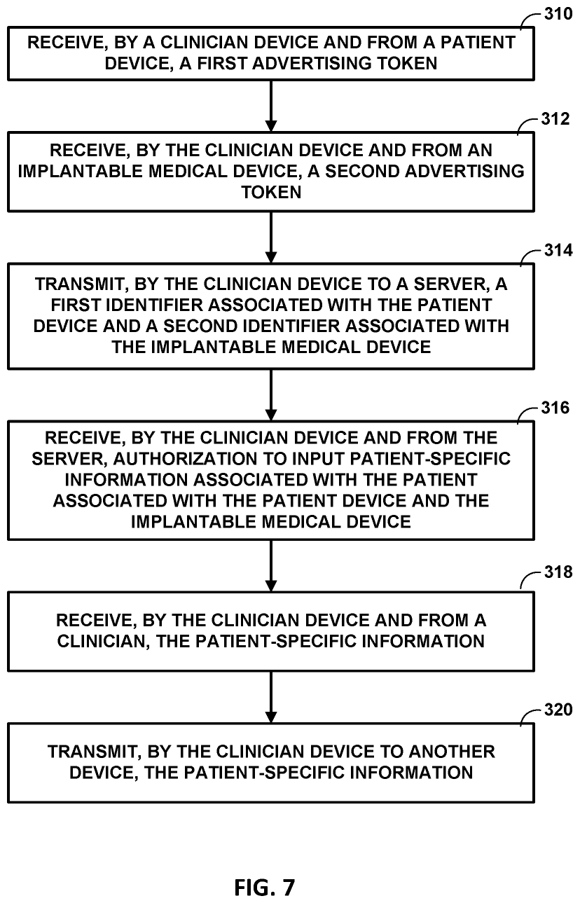

FIG. 7 is a flowchart illustrating another example of configuration techniques in accordance with this disclosure. Clinician device 104 may receive a first advertising token from patient device 102 via communication circuitry 210 (310). For example, clinician device 104 may receive the first advertising token from patient device 102 that patient device received from server 94 via communication circuitry 210. Clinician device 104 may receive a second advertising token from IMD 16 via communication circuitry 210 (312). For example, IMD 16 may advertise the second advertising token via communication circuitry 17 and clinician device 104 may receive the second advertising token via communication circuitry 210.

Clinician device 104 may transmit a first identifier associated with patient device 102 and a second identifier associated with IMD 16 to server 94 via communication circuitry 210 (314). For example, clinician device 104 may transmit a UUID associated with patient device 102 and a UUID and/or SDN associated with IMD 16 to server 94 via communication circuitry 210.

Clinician device 104 may receive authorization to input patient-specific information associated with patient 14 associated with patient device 102 and IMD 16 from server 94 via communication circuitry 210 (316). For example, server 94 may verify that the UUID from patient device 102 is authorized and associate IMD 16 with patient device 102. Server 94 may send an authorization to clinician device 104 to enable clinician device 104 to enter patient-specific information in an instance of the monitoring application running on clinician device 104.

Clinician device 104 may receive, from a clinician, the patient-specific information via user interface 208 (of FIG. 3) (318). For example, a clinician may enter the patient-specific information into an instance of the monitoring application running on clinician device 104 via user interface 208.

Clinician device 104 may transmit, to another device, the patient-specific information via communication circuitry 210 (320). In some examples, clinician device 104 may transmit the patient-specific information to server 94 and server 94 may push the patient-specific information to patient device 102. In other examples, clinician device 104 may connect with patient device 102 and push the patient-specific information to patient device 102 directly via communication circuitry 210. For example, clinician device 104 may connect with patient device 102 via a personal area networking technology.

In this manner, a clinician may configure a monitoring application on a patient device and/or create a user account with a remote monitoring service for a patient without physically contacting the patient device. As such, the techniques of this disclosure may reduce liability risk to a clinician and improve the data integrity of the patient-specific information.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

The techniques described in this disclosure may also be embodied or encoded in a non-transitory computer-readable medium, such as a computer-readable storage medium, containing instructions. Instructions embedded or encoded in a non-transitory computer-readable storage medium may cause a programmable processor, or other processor, to perform the method, e.g., when the instructions are executed. Non-transitory computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

This disclosure contains the following non-limiting examples.

Example 1. A method comprising: receiving, by a patient device, an advertising token from a server; wirelessly advertising for a connection, by the patient device, using the advertising token; in response to the wirelessly advertising, receiving, by the patient device and from another device, patient-specific information relating to a patient associated with the patient device; and configuring, by the patient device, a monitoring application to include the patient-specific information.

Example 2. The method of claim 1, wherein the another device is the server.

Example 3. The method of claim 1, wherein the another device is a clinician device, and the method further comprises: connecting, by the patient device, with the clinician device.

Example 4. The method of claim 3, wherein the connecting is via a personal area networking technology.

Example 5. The method of claim 1, further comprising: downloading, by patient device, the monitoring application from the server.

Example 6. The method of claim 1, further comprising: registering, by the patient device, with the server.

Example 7. The method of claim 1, wherein the advertising token is a Bluetooth low energy (BLE) peripheral token.

Example 8. A patient device comprising: memory configured to store a monitoring application; communication circuitry configured to communicate with at least one of a server or another device; and processing circuitry communicatively coupled to the memory and the communication circuitry, the processing circuitry being configured to: receive an advertising token from the server via the communication circuitry; wirelessly advertise for a connection using the advertising token via the communication circuitry; in response to the wirelessly advertising, receive patient-specific information relating to a patient associated with the patient device from the another device via the communication circuitry; and configure the monitoring application to include the patient-specific information.

Example 9. The patient device of claim 8, wherein the another device is the server.

Example 10. The patient device of claim 8, wherein the another device is a clinician device, and the processing circuitry is further configured to: connect with the clinician device via the communication circuitry.

Example 11. The patient device of claim 10, wherein the connecting is via a personal area networking technology.

Example 12. The patient device of claim 8, wherein the processing circuitry is further configured to: download the monitoring application from the server via the communication circuitry.

Example 13. The patient device of claim 8, wherein the processing circuitry is further configured to: register with the server via the communication circuitry.

Example 14. The patient device of claim 8, wherein the advertising token is a Bluetooth low energy (BLE) peripheral token.

Example 15. A method comprising: receiving, by a clinician device and from a patient device, a first advertising token; receiving, by the clinician device and from an implantable medical device, a second advertising token; transmitting, by the clinician device to a server, a first identifier associated with the patient device and a second identifier associated with the implantable medical device; receiving, by the clinician device from the server, authorization to input patient-specific information associated with a patient associated with the patient device and the implantable medical device; receiving, by the clinician device from a clinician, the patient-specific information; and transmitting, by the clinician device to another device, the patient-specific information.

Example 16. The method of claim 15, wherein the another device is the server.

Example 17. The method of claim 15, wherein the another device is a patient device, and the method further comprises: connecting with the patient device.

Example 18. A clinician device comprising: memory configured to store a monitoring application; communication circuitry configured to communicate with at least one of a server or another device; a user interface configured to accept input from a user; and processing circuitry communicatively coupled to the memory, the communication circuitry and the user interface, the processing circuitry being configured to: receive a first advertising token from a patient device via the communication circuitry; receive a second advertising token from an implantable medical device via the communication circuitry; transmit a first identifier associated with the patient device and a second identifier associated with the implantable medical device to a server via the communication circuitry; receive authorization to input patient-specific information associated with a patient associated with the patient device and the implantable medical device from the server via the communication circuitry; receive, from a clinician, the patient-specific information, via the user interface; and transmit, to the another device, the patient-specific information via the communication circuitry.

Example 19. The clinician device of claim 18, wherein the another device is the server.

Example 20. The clinician device of claim 18, wherein the another device is a patient device, and the processing circuitry is further configured to: connect with the patient device.

Various examples have been described. These and other examples are within the scope of the following claims.

What is claimed is:

1. A method comprising:
receiving, by a patient device, an advertising token from a server;
wirelessly advertising for a connection, by the patient device, using the advertising token;
in response to the wirelessly advertising, receiving, by the patient device and from another device, patient-specific information relating to a patient associated with the patient device; and
configuring, by the patient device, a monitoring application to include the patient-specific information.

2. The method of claim 1, wherein the another device is the server.

3. The method of claim 1, wherein the another device is a clinician device, and the method further comprises:
connecting, by the patient device, with the clinician device.

4. The method of claim 3, wherein the connecting is via a personal area networking technology.

5. The method of claim 1, further comprising:
downloading, by patient device, the monitoring application from the server.

6. The method of claim 1, further comprising:
registering, by the patient device, with the server.

7. The method of claim 1, wherein the advertising token is a Bluetooth low energy (BLE) peripheral token.

8. A patient device comprising:
memory configured to store a monitoring application;
communication circuitry configured to communicate with at least one of a server or another device; and
processing circuitry communicatively coupled to the memory and the communication circuitry, the processing circuitry being configured to:
receive an advertising token from the server via the communication circuitry;
wirelessly advertise for a connection using the advertising token via the communication circuitry;
in response to the wirelessly advertising, receive patient-specific information relating to a patient associated with the patient device from the another device via the communication circuitry; and
configure the monitoring application to include the patient-specific information.

9. The patient device of claim 8, wherein the another device is the server.

10. The patient device of claim 8, wherein the another device is a clinician device, and the processing circuitry is further configured to:
connect with the clinician device via the communication circuitry.

11. The patient device of claim 10, wherein the connecting is via a personal area networking technology.

12. The patient device of claim 8, wherein the processing circuitry is further configured to:
download the monitoring application from the server via the communication circuitry.

13. The patient device of claim 8, wherein the processing circuitry is further configured to:
register with the server via the communication circuitry.

14. The patient device of claim 8, wherein the advertising token is a Bluetooth low energy (BLE) peripheral token.

15. A method comprising:
receiving, by a clinician device and from a patient device, a first advertising token;
receiving, by the clinician device and from an implantable medical device, a second advertising token;
transmitting, by the clinician device to a server, a first identifier associated with the patient device and a second identifier associated with the implantable medical device;
receiving, by the clinician device from the server, authorization to input patient-specific information associated with a patient associated with the patient device and the implantable medical device;
receiving, by the clinician device from a clinician, the patient-specific information; and
transmitting, by the clinician device to another device, the patient-specific information.

16. The method of claim 15, wherein the another device is the server.

17. The method of claim 15, wherein the another device is a patient device, and the method further comprises:
connecting with the patient device.

18. A clinician device comprising:
memory configured to store a monitoring application;
communication circuitry configured to communicate with at least one of a server or another device;
a user interface configured to accept input from a user; and
processing circuitry communicatively coupled to the memory, the communication circuitry and the user interface, the processing circuitry being configured to:
receive a first advertising token from a patient device via the communication circuitry;
receive a second advertising token from an implantable medical device via the communication circuitry;
transmit a first identifier associated with the patient device and a second identifier associated with the implantable medical device to a server via the communication circuitry;
receive authorization to input patient-specific information associated with a patient associated with the patient device and the implantable medical device from the server via the communication circuitry;
receive, from a clinician, the patient-specific information, via the user interface; and
transmit, to the another device, the patient-specific information via the communication circuitry.

19. The clinician device of claim 18, wherein the another device is the server.

20. The clinician device of claim 18, wherein the another device is a patient device, and the processing circuitry is further configured to:
connect with the patient device.
